(19) United States
(12) Patent Application Publication
(10) Pub. No.: US 2012/0045760 A1
(43) Pub. Date: Feb. 23, 2012

(54) Single Nucleotide Polymorphism Within An Intronic P53 Binding Motif of the Prkag2 Gene

(75) Inventors: Jianjun Liu, Singapore (SG); Tak-Bun Edison Liu, Singapore (SG); Heli Nevanlinna, Helsinki (FI); Per Hall, Stockholm (SE)

(73) Assignees: AGENCY FOR SCIENCE, TECHNOLOGY, AND RESEARCH, Singapore (SG); CLINICAL RESEARCH INSTITUTE HELSINKI UNIVERSITY CENTRAL HOSPITAL, Helsinki (FI); KAROLINSKA INSTITUTET, Stockholm (SE)

(21) Appl. No.: 13/145,874

(22) PCT Filed: Jan. 22, 2010
(86) PCT No.: PCT/SG10/00020

§ 371 Date: Jul. 22, 2011

Related U.S. Application Data
(60) Provisional application No. 61/147,030, filed on Jan. 23, 2009.

(51) Int. Cl. C12Q 1/68; G01N 33/566; C12Q 1/02; C12N 15/11; C12N 15/63

(52) U.S. Cl. 435/6.11; 536/23.1; 435/320.1; 435/29; 436/501

(57) ABSTRACT

The present invention relates to single nucleotide polymorphism (SNP). In particular, it relates to a SNP within an intronic p53 binding motif of the PRKAG2. Nucleic acid molecules and methods for aiding assessment of a patient's risk of developing cancer by determining the patient's genotype for a p53 binding motif within the PRKAG2 gene are included in the present invention.

P53 is best known as a tumor suppressor gene that is also mechanistically involved in DNA repair (Vousden and Lu 2002). When activated in response to stress signals, p53 can trigger multiple cellular processes including cell-cycle arrest, senescence and apoptosis. Recent data have shown that p53 plays a broader role as the tumor suppressor gene and might be involved in other biological processes such as metabolism but the molecular mechanisms of this involvement are not well understood (Vousden and Lane 2007). p53-mediated cellular responses are mainly achieved through the transcriptional regulation of p53 downstream target genes where p53 functions as a nuclear transcription factor, although transcriptionally independent mechanisms have also been demonstrated for p53 (Chipuk, Maurer et al. 2003; Chipuk, Kuwana et al. 2004; Tan, Zhuang et al. 2005). Given the important and broad role of p53 protein, it has been of great interest to identify the target genes of p53's transcriptional regulation. Earlier efforts using gene expression profiling analyses (Yu, Zhang et al. 1999; Zhao, Gish et al. 2000; Kannan, Kaminski et al. 2001; Kho, Wang et al. 2004) have identified many genes to be putatively downstream of the p53 protein, but these studies could not distinguish between the direct target genes and the secondary targets of p53 or identified genomic sequences that mediate p53′s transcriptional regulation.

The DNA binding sites of p53 on a genome-wide scale have been assessed using technologies like chromatin immunoprecipitation (ChIP) followed by hybridization to an array chip (ChIP-Chip) or by shotgun sequencing of ChIP pull-down DNA fragments (ChIP-seq or ChIP-PET for Pair-End diTag reads) (Cawley, Bekiranov et al. 2004; Wei, Wu et al. 2006). With these approaches, putative p53 direct target genes could be computationally imputed by proximity of regulated genes near bona fide binding sites, and the likely regulatory sites can be identified in a genome wide basis. Of particular importance is the observation that these bona fide binding sites are distributed widely around and within genes (+/−100 kb from the transcription start site, TSS, or the polyadenylation signal sequence). Any pure computation means of predicting functional p53 binding sites classically by a 5 kb proximity to the TSS would have had a 95% error rate. We showed that these experimentally determined p53 binding sites were likely to be involved in direct transcriptional regulation since expression profiles of genes thus identified could distinguish tumors that are mutant in p53 from those that were wild-type (Wei, Wu et al. 2006).

By mining the public dbSNP database, a number of these binding sites harbor sequence polymorphisms have been observed either in close proximity to or directly within the p53 binding motifs which could potentially lead to altered p53 DNA binding. Some of these allelic variants will be associated with differential p53 binding and even differential gene expression of p53 target genes. To date, the genetic and molecular analyses of p53-related regulatory sequence variants in populations have been limited (Pietsch, Humbey et al. 2006), largely due to the lack of experimental means to identify true (rather than computationally predicted) p53-related regulatory sequences. The most intensively studied regulatory variant within the p53 pathway is the T/G polymorphism within the intronic promoter of MDM2, a strong negative regulator of p53 protein activity. The polymorphism was shown to increase the binding affinity of the transcription activator Sp1 and thus the levels of MDM2 RNA and protein, which further results in decreased level of p53 protein and accelerated tumor formation in humans (Bond, Hu et al. 2004). A series of the subsequent genetic analyses of this polymorphism, however, failed to provide consistent evidence. Recently, a meta-analysis of 21 case-control studies showed that the homozygous genotype of the minor allele variant is associated with an increased risk for cancer development, especially of lung cancer and smoking-related cancers (Hu, Jin et al. 2007), but no such evidence has been shown for breast cancer although very low risk effect may not be excluded (Schmidt, Reincke et al. 2007). Similarly, Mendendez et al identified a C-to-T polymorphism within the proximal promoter region of the fit-1gene, where the minor allele of T created a half-binding site for p53. This brought the system under the control of p53 network (Menendez, Inga et al. 2007). Interestingly, a recent effort by the same group has further demonstrated that the presence of this polymorphism also created a partial responsible element for estrogen receptor upstream the previous identified half-binding site for p53, which introduces a mechanism for synergistic simulation of transcription at this fit-promoter site through the combined action of p53 and ER (Menendez, Inga et al. 2007). The genome-wide identification of p53 binding sequences by ChIP-ChIP and ChIP-PET analyses expands the possibilities for directly investigating the molecular and physiological functions of genetic variation within these binding sequences.

Known SNPs within a group of the p53 binding sites identified by a genome-wide ChIP-PET mapping analysis of p53 have been searched (Wei, Wu et al. 2006). A common SNP within an intronic p53 binding motif in the third intron of PRKAG2 has been identified. By performing a series of genetic and functional analyses, it is demonstrated that the homozygous genotypes of the minor allele at this SNP locus can significantly reduce the binding affinity of p53 protein to this binding site and the down-regulation of the target gene PRAKG2 mRNA expression and the AMPK protein complex. By genotyping this SNP in three samples of breast and endometrial cancers, it is further demonstrated that the genotype of the SNP can significantly influence the susceptibility to cancer development.

It is currently believed that the p53 binding motif is within the third intron of the PRKAG2 gene.

In accordance with a first aspect of the invention, there is provided an isolated nucleic acid molecule of less than 100, 50 or 30 nucleotides or base pairs comprising the sequence 5′ RRRCWWGYYYRRRCWWTYYY-3′ or its complement.

By “isolated”, it is meant that the nucleic acid is not located in a cell, i.e. in situ, but is suitable for in vitro use in the methods of the invention.

Preferably, the isolated nucleic acid molecule of claim 1 that is capable of hybridising to or having at least 50, 60, 70, 80, 90 or 95% identity with, the region encompassing the p53 binding motif within the third intron of the PRKAG2 gene or its complement that can be amplified from human genomic DNA using the PCR primers 5′-TAGGAGACCTGGGGGACTTT-3′ and 5′-CAGGCATCTCGAAGAGATCA-3′. Preferably, the sequence of this region fragment (142 bp) is:

CCATCCTGCCTGAGCATGTCTGAACatgttcttaggtcaggactagagttcgagatttcagaaatgtcattc
taaccttgatctettcgagatgcctgtttataacacagcatcgttcatgCCAATTGTCTGGCAAAGCCGG

Analysis of sequence loci can be by methods such as Southern blot analysis, conventional PCR amplification. See, e.g., Innis et al., PCR Strategies (Academic Press, Inc.: NY., 1995); Dieffenbach et al., PCR Primer. A Laboratory Manual (New York: Cold Spring Harbor Press, 1995), denaturing gradient gel-electrophoresis (Myers, et al., 1987. Meth. Enzymol. 155: 501), single-strand conformational analysis (Hayashi, 1992. Genet Anal Biomol E 9: 73), ligase-chain reaction (Barany. 1991. Proc Natl Acad Sci 88: 189), isothermal amplification (Fahy et al. 1991. PCR Methods Appl 1: 25), branched chain analysis (Urdea. 1993. Clin Chem 39: 725), and signal amplification techniques such as Third Wave's linear amplification. DNA sequence analysis may also be achieved by detecting alteration in electrophoretic mobility of DNA fragments in gels with or without denaturing agents. Samples containing sequence insertions can also be visualized by high resolution gel electrophoresis or distinguished according to differences in DNA sequence melting points. See, e.g., Myers et al., Science, 230: 1242 (1982). Methods for detecting presence of specific sequences include detection techniques such as fluorescence-based detection methods, immune-based assays such as RIA, antibody staining such as Western blot analysis or in situ hybridization, using appropriately labeled probe.

Sequences useful for constructing probes suitable for use in detecting presence of a sequence of interest include any nucleic acid sequence having at least about 80% or greater sequence identity or homology with the sequence by a Blast search. “Percent (%) sequence identity” or “percent (%) sequence homology” is defined as the percentage of nucleic acid residues in a candidate sequence that are identical with the nucleic acid residues of the sequence of interest, after aligning the sequences and introducing gaps, if necessary to achieve maximum percent sequence identity, and not considering any conservative substitutions as part of the sequence identity. Methods for performing sequence alignment and determining sequence identity are known in the art, may be performed without undue experimentation, and calculations of % identity values may be obtained for example, using available computer programs such as WU-BLAST-2 (Altschul et al., Methods in Enzymology 266:460-480 (1996). One may optionally perform the alignment using set default parameters in the computer software program (Blast search,MacVector and Vector NTI).

Based upon the restriction map of a particular locus, a banding pattern can be predicted when the Southern blot is hybridized with a probe which recognizes the sequence of interest. The level of stringency of hybridization used can vary depending upon the level of sensitivity desired, a particular probe characteristic, such as probe length and/or annealing temperature, or degree of homology between probe sequence and sequence of interest. Therefore, considerations of sensitivity and specificity will determine stringency of hybridization required for a particular assay.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to re-anneal when complementary strands are present in an environment below their melting temperatures. The higher the degree of desired homology between the probe and hybridizable sequence, the higher the relative temperature that can be used. For additional details and explanation of stringency of hybridization reactions, see Ausubel et al. Current Protocols in Molecular Biology (Wiley Interscience Publishers, 1995) or Protocols Online URL: www.protocol-online.net/molbio/index.htm).

DNA-DNA, DNA-RNA and RNA-RNA hybridisation may be performed in aqueous solution containing between 0.1×SSC and 6×SSC and at temperatures of between 55° C. and 70° C. It is well known in the art that the higher the temperature or the lower the SSC concentration the more stringent the hybridisation conditions. By “high stringency”, it means 2×SSC and 65° C. 1×SSC is 0.15M NaCl/0.015M sodium citrate. Polynucleotides which hybridise at high stringency are included within the scope of the claimed invention.

It is meant that the nucleic acid has sufficient nucleotide sequence similarity with the said p53 binding motif within the third intron of the PRKAG2 gene or its complement that it can hybridise under moderately or highly stringent conditions. As is well known in the art, the stringency of nucleic acid hybridization depends on factors such as length of nucleic acid over which hybridisation occurs, degree of identity of the hybridizing sequences and on factors such as temperature, ionic strength and CG or AT content of the sequence. Thus, any nucleic acid which is capable of hybridising as said is useful in the practice of the invention.

Nucleic acids which can selectively hybridise to the said p53 binding motif include nucleic acids which have 50% sequence identity, preferably those with 60%, more preferably those with 70% sequence identity, still more preferably those with 80% sequence identity, still more preferably those with 90% sequence identity, still more preferably those with 95% sequence identity, over at least a portion of the nucleic acid with the said nucleic acid.

Typical moderately or highly stringent hybridisation conditions which lead to selective hybridisation are known in the art, for example those described in Molecular Cloning, a laboratory manual, 2nd edition, Sambrook et al (eds), Cold Spring Harbor Laboratory Press, Cold Spring Harbor, N.Y., USA, incorporated herein by reference.

An example of a typical hybridisation solution when a nucleic acid is immobilised on a nylon membrane and the probe nucleic acid is z 500 bases or base pairs is: 6×SSC (saline sodium citrate) 0.5% sodium dodecyl sulphate (SDS) 100 g/ml denatured, fragmented salmon sperm DNA The hybridisation is performed at 68° C. The nylon membrane, with the nucleic acid immobilised, may be washed at 68° C. in 1×SSC or, for high stringency, 0.1×SSC.

20×SSC may be prepared in the following way. Dissolve 175.3 g of NaCl and 88.2 g of sodium citrate in 800 ml of H20. Adjust the pH to 7.0 with a few drops of a 10 N solution of NaOH. Adjust the volume to 1 litre with H20. Dispense into aliquots. Sterilize by autoclaving.

Suitable conditions for PCR amplification include amplification in a suitable 1× amplification buffer: 10× amplification buffer is 500 mM KCl; 100 mM Tris. Cl (pH 8.3 at room temperature); 15 mM MgCl2; 0.1% gelatin. A suitable denaturing agent or procedure (such as heating to 95° C.) is used in order to separate the strands of double-stranded DNA. Suitably, the annealing part of the amplification is between 37° C. and 60° C., preferably 50° C.

Although the nucleic acid which is useful in the methods of the invention may be RNA or DNA, DNA is preferred. Although the nucleic acid which is useful in the methods of the invention may be double-stranded or single-stranded, single-stranded nucleic acid is preferred under some circumstances such as in nucleic acid amplification reactions.

Single-stranded DNA primers, suitable for use in a polymerase chain reaction, are particularly preferred. The nucleic acid for use in the methods of the invention is a nucleic acid which hybridises to p53 binding motiff. cDNAs derivable from the p53 binding motiff are preferred nucleic acids for use in the methods of the invention.

Primers which are suitable for use in a polymerase chain reaction (PCR; Saiki et al (1988) Science 239,487-491) are preferred. Suitable PCR primers may have the following properties: It is well known that the sequence at the 5′end of the oligonucleotide need not match the target sequence to be amplified.

It is usual that the PCR primers do not contain any complementary structures with each other longer than 2 bases, especially at their 3′ends, as this feature may promote the formation of an artifactual product called “primer dimer”. When the 3′ends of the two primers hybridize, they form a “primed template” complex, and primer extension results in a short duplex product called “primer dimer”.

Internal secondary structure should be avoided in primers. For symmetric PCR, a 40-60% G+C content is often recommended for both primers, with no long stretches of any one base. The classical melting temperature calculations used in conjunction with DNA probe hybridization studies often predict that a given primer should anneal at a specific temperature or that the 72° C. extension temperature will dissociate the primer/template hybrid prematurely. In practice, the hybrids are more effective in the PCR process than generally predicted by simple Tm calculations.

Optimum annealing temperatures may be determined empirically and may be higher than predicted. Taq DNA polymerase does have activity in the 37-55° C. region, so primer extension will occur during the annealing step and the hybrid will be stabilized. The concentrations of the primers are equal in conventional (symmetric) PCR and, typically, within 0.1- to 1-,range.

Any of the nucleic acid amplification protocols can be used in the method of the invention including the polymerase chain reaction, QB replicase and ligase chain reaction. Also, NASBA (nucleic acid sequence based amplification), also called 3SR, can be used as described in Compton (1991) Nature 350,91-92 and AIDS (1993), Vol 7 (Suppl 2), S108 or SDA (strand displacement amplification) can be used as described in Walker et al (1992) Nucl. Acids Res. 20,1691-1696. The polymerase chain reaction is particularly preferred because of its simplicity.

When a pair of suitable nucleic acids of the invention is used in a PCR it is convenient to detect the product by gel electrophoresis and ethidium bromide staining. As an alternative to detecting the product of DNA amplification using agarose gel electrophoresis and ethidium bromide staining of the DNA, it is convenient to use a labelled oligonucleotide capable of hybridising to the amplified DNA as a probe. When the amplification is by a PCR the oligonucleotide probe hybridises to the interprimer sequence as defined by the two primers. The oligonucleotide probe is preferably between 10 and 50 nucleotides long, more preferably between 15 and 30 nucleotides long. The probe may be labelled with a radionuclide such as ³²P, ³³P and ³⁵S using standard techniques, or may be labelled with a fluorescent dye. When the oligonucleotide probe is fluorescently labelled, the amplified DNA product may be detected in solution (see for example Balaguer et al (1991) “Quantification of DNA sequences obtained by polymerase chain reaction using a bioluminescence adsorbent” Anal. Biochem. 195,105-110 and DiCesare et al (1993) “A high-sensitivity electrochemiluminescence-based detection system for automated PCR product quantitation “BioTechniques 15,152-157.

Amplification products can also be detected using a probe which may have a fluorophore-quencher pair or may be attached to a solid support or may have a biotin tag or they may be detected using a combination of a capture probe and a detector probe.

Fluorophore-quencher pairs are particularly suited to quantitative measurements of PCR reactions (eg RT-PCR). Fluorescence polarisation using a suitable probe may also be used to detect PCR products.

In accordance with a second aspect of the invention, there is provided an isolated nucleic acid molecule of between 25, 50 or 100 and 300 nucleotides or base pairs comprising the sequence 5′ RRRCWWGYYYRRRCWWTYYY-3′ or 5′ RRRCWWGYYYRRRCWWGYYY-3′ and capable of hybridising to or having at least 50, 60, 70, 80, 90 or 95% identity with, the region encompassing the p53 binding motif within the third intron of the PRKAG2 gene or its complement that can be amplified from human genomic DNA using the PCR primers 5′-TAGGAGACCTGGGGGACTTT-3′ and 5′-CAGGCATCTCGAAGAGATCA-3′. Preferably, the sequence of this region fragment (226 bp) is:

TAGGAGACCTGGGGGACTTTcatactctcagctgatgccagggtgcccagtgagcaggggaaaggcttcc
tggccctggcggcaggatggggccagaatattcctgggcaggagcccccccaggtggcccatcctgcctg
agcatgtctgaacatgttcttaggtcaggactagagttcgagatttcagaaatgtcattctaacct
TGATCTCTTCGAGATGCCTG

In accordance with a third aspect of the invention, there is provided an isolated nucleic acid having the sequence 5′-TAGGAGACCTGGGGGACTTT-3′; 5′-CAGGCATCTCGAAGAGATCA-3; 5′-CCATCCTGCCTGAGCATGTCTGAAC; or CCGGCTTTGCCAGACAATTGG.

In accordance with a fourth aspect of the invention, there is provided a vector comprising a nucleic acid according to the first, second and third aspects of the invention.

It would be understood by someone skilled in the art of molecular biology that many vectors and packaging cell lines are available for delivering the nucleic acids that could be used for treatment.

Typical prokaryotic vector plasmids are: pUC18, pUC19, pBR322 and pBR329 available from Biorad Laboratories (Richmond, Calif., USA); pTrc99A, pKK223-3, pKK233-3, pDR540 and pRIT5 available from Pharmacia (Piscataway, N.J., USA); pBS vectors, Phagescript vectors, Bluescript vectors, pNH8A, pNH16A, pNH18A, pNH46A available from Stratagene Cloning Systems (La Jolla, Calif. 92037, USA).

A typical mammalian cell vector plasmid is pSVL available from Pharmacia (Piscataway, N.J., USA). This vector uses the SV40 late promoter to drive expression of cloned genes, the highest level of expression being found in T antigen-producing cells, such as COS-1 cells. An example of an inducible mammalian expression vector is pMSG, also available from Pharmacia (Piscataway, N.J., USA). This vector uses the glucocorticoid-inducible promoter of the mouse mammary tumour virus long terminal repeat to drive expression of the cloned gene.

Useful yeast plasmid vectors are pRS403-406 and pRS413-416 and are generally available from Stratagene Cloning Systems (La Jolla, Calif. 92037, USA). Plasmids pRS403, pRS404, pRS405 and pRS406 are Yeast Integrating plasmids (Yips) and incorporate the yeast selectable markers HIS3, TRP1, LEU2 and URA3. Plasmids pRS413-416 are Yeast Centromere plasmids (YCps).

Methods well known to those skilled in the art can be used to construct expression vectors containing the coding sequence and, for example appropriate transcriptional or translational controls. One such method involves ligation via homopolymer tails. Homopolymer polydA (or polydC) tails are added to exposed 3′ OH groups on the DNA fragment to be cloned by terminal deoxynucleotidyl transferases. The fragment is then capable of annealing to the polydT (or polydG) tails added to the ends of a linearised plasmid vector. Gaps left following annealing can be filled by DNA polymerase and the free ends joined by DNA ligase.

Another method involves ligation via cohesive ends. Compatible cohesive ends can be generated on the DNA fragment and vector by the action of suitable restriction enzymes. These ends will rapidly anneal through complementary base pairing and remaining nicks can be closed by the action of DNA ligase.

A further method uses synthetic molecules called linkers and adaptors. DNA fragments with blunt ends are generated by bacteriophage T4 DNA polymerase or E.coli DNA polymerase I which remove protruding 3′ termini and fill in recessed 3′ ends. Synthetic linkers, pieces of blunt-ended double-stranded DNA which contain recognition sequences for defined restriction enzymes, can be ligated to blunt-ended DNA fragments by T4 DNA ligase. They are subsequently digested with appropriate restriction enzymes to create cohesive ends and ligated to an expression vector with compatible termini. Adaptors are also chemically synthesised DNA fragments which contain one blunt end used for ligation but which also possess one preformed cohesive end.

Synthetic linkers containing a variety of restriction endonuclease sites are commercially available from a number of sources including International Biotechnologies Inc, New Haven, Conn., USA.

A desirable way to modify the DNA encoding the polypeptide of the invention is to use the polymerase chain reaction as disclosed by Saiki et al (1988) Science 239, 487-491. In this method the DNA to be enzymatically amplified is flanked by two specific oligonucleotide primers which themselves become incorporated into the amplified DNA. The said specific primers may contain restriction endonuclease recognition sites which can be used for cloning into expression vectors using methods known in the art.

In accordance with a fifth aspect of the invention, there is provided a molecule comprising a nucleic acid molecule according to the first, second, third and fourth aspects of the invention, and a detectable moiety. For example, they may be labelled in such a way that they may be directly or indirectly detected.

Preferably, the detectable moiety is a fluorophore or a radioisotope.

Conveniently, the polynucleotides are labelled with a radioactive moiety or a coloured moiety or a fluorescent moiety or some other suitable detectable moiety such as digoxygenin and luminescent or chemiluminescent moieties. The polynucleotides may be linked to an enzyme, or they may be linked to biotin (or streptavidin) and detected in a similar way as described for antibodies of the invention. Also preferably the polynucleotides of the invention may be bound to a solid support (including arrays, beads, magnetic beads, sample containers and the like).

The nucleic acids of the invention may also incorporate a “tag” nucleotide sequence which tag sequence can subsequently be recognised by a further nucleic acid probe. Suitable labels or tags may also be used for the selective capture of the hybridised (or non-hybridised) polynucleotide using methods well known in the art.

Preferably, the nucleic acid may be used in diagnosis.

In accordance with a sixth aspect of the invention, there is provided a method for aiding assessment of a patient's risk of developing cancer, or likely severity or likelihood of progression of cancer, or aiding in selection of a cancer treatment regime for the patient, or aiding in assessment of a cancer treatment regime, the method comprising determining the patient's genotype for a p53 binding motif within the PRKAG2 gene.

Preferably, the p53 binding motif is within the third intron of the PRKAG2 gene.

Preferably, the method comprises determining the presence or absence of a single nucleotide polymorphism relative to the wild-type p53 binding motif within the third intron of the PRKAG2 gene.

Preferably, the wild-type p53 binding motif within the third intron of the PRKAG2 gene comprises the sequence 5′-RRRCWWGYYYRRRCWWGYYY-3′ and can be amplified from human genomic DNA using the PCR primers 5′-TAGGAGACCTGGGGGACTTT-3′ and 5′-CAGGCATCTCGAAGAGATCA-3′.

Preferably, the single nucleotide polymorphism is the substitution of the G residue marked with a * within the sequence 5′ RRRCWWGYYYRRRCWWG*YYY-3′, for example by a T residue.

Preferably, the single nucleotide polymorphism is at locus rs1860746 of the dbSNP public database.

Preferably, sequencing, primer extension, allele-specific PCR or TaqMan assay is used in determining the patient's genotype for a p53 binding motif within the PRKAG2 gene.

In accordance with a seventh aspect of the invention, there is provided a method for aiding assessment of a patient's risk of developing cancer, or likely severity or likelihood of progression of cancer, or aiding in selection of a cancer treatment regime for the patient, or aiding in assessment of a cancer treatment regime, the method comprising determining the patient's AMPK protein levels, phosphorylation levels, catalytic activity or mRNA levels.

It will be appreciated that detecting the presence of a decreased level of AMPK protein levels in a cell compared to the level present in a normal (non-cancerous) cell may aid in the assessment of a patient's risk of developing cancer.

The RNA levels of AMPK may be determined by using specific oligonucleotide primers and a nucleic acid amplification technique such as the polymerase chain reaction (PCR). Oligonucleotide primers can be synthesised using methods well known in the art, for example using solid-phase phosphoramidite chemistry. Preferably, the oligonucleotide primers are at least 20 nucleotides in length, more preferably at least 25 nucleotides in length and still more preferably at least 29 nucleotides in length.

Suitable conditions for PCR amplification include amplification in a suitable 1× amplification buffer: 10× amplification buffer is 500 mM KCl; 100 mM Tris. Cl (pH 8.3 at room temperature); 15 mM MgCl2; 0. 1% gelatin. single-stranded DNA primers, suitable for use in a polymerase chain reaction, are particularly preferred.

It will be appreciated that AMPK mRNA may be identified by reverse-transcriptase polymerase chain reaction (RT-PCR) using methods well known in the art.

Other methods of detecting mRNA levels are included.

Methods for determining the relative amount of AMPK mRNA include: in situ hybridisation (In Situ Hybridization Protocols. Methods in Molecular Biology Volume 33. Edited by K H A Choo. 1994, Humana Press Inc (Totowa, N.J., USA) pp 480p and In Situ Hybridization: A Practical Approach. Edited by D G Wilkinson. 1992, Oxford University Press, Oxford, pp 163), in situ amplification, northems, nuclease protection, probe arrays, and amplification based systems; The mRNA may be amplified prior to or during detection and quantitation.'Real time' amplification methods wherein the product is measured for each amplification cycle may be particularly useful (eg Real time PCR Hid et al (1996) Genome Research 6,986-994, Gibson et at (1996) Genome Research 6,995-1001; Real time NASBA Oehlenschlager et at (1996 Nov. 12) PNAS (USA) 93 (23), 12811-6. Primers should be designed to preferentially amplify from an mRNA template rather than from the DNA, or be designed to create a product where the mRNA or DNA template origin can be distinguished by size or by probing. NASBA may be particularly useful as the process can be arranged such that only RNA is recognised as an initial substrate.

Detecting mRNA includes detecting mRNA in any context, or detecting that there are cells present which contain mRNA (for example, by in situ hybridisation, or in samples obtained from lysed cells). It is useful to detect the presence of mRNA or that certain cells are present (either generally or in a specific location) which can be detected by virtue of their expression of AMPK mRNA. As noted, the presence versus absence of AMPK mRNA may be a useful marker, or low levels versus high levels of AMPK mRNA may be a useful marker, or specific quantified levels may be associated with a specific disease state. It will be appreciated that similar possibilities exist in relation to using the AMPK polypeptide as a marker.

Alternatively, the method further comprises determining the protein levels of AMPK in the sample.

The methods of the invention also include the measurement and detection of the AMPK polypeptide in test samples and their comparison in a reference sample.

The sample containing RNA and/or protein derived from the patient is conveniently a sample of the tissue in which cancer is suspected or in which cancer may be or has been found. These methods may be used for any cancer, but they are particularly suitable in respect of breast or endometrial cancers. The sample may also be blood, serum or lymph nodes which may be particularly useful in determining whether a cancer has spread. Alternatively, the sample may be tissue sample obtained surgically from a patient.

The methods of the invention involving detection of the AMPK polypeptide are particularly useful in relation to historical samples such as those containing paraffin-embedded sections of tumour samples.

The amount of the AMPK polypeptide may be determined in any suitable way.

It is preferred if the amount of the AMPK polypeptide is determined using a molecule which selectively binds to AMPK polypeptide. Suitably, the molecule which selectively binds to AMPK may be an antibody. The antibody may also bind to a natural variant or fragment of AMPK polypeptide.

By “variants” of the polypeptide we include insertions, deletions and substitutions, either conservative or non-conservative, where such changes do not substantially alter the activity of the said AMPK.

Variants and variations of the polynucleotide and polypeptide include natural variants, including allelic variants and naturally-occurring mutant forms.

By “fragment of AMPK”, we include any fragment which retains activity or which is useful in some other way, for example, for use in raising antibodies or in a binding assay.

The antibodies for use in the methods of the in invention may be monoclonal or polyclonal.

The protein levels of AMPK may be determined using any suitable protein quantitation method. In particular, it is preferred if antibodies are used and that the amount of AMPK is determined using methods which include quantititative western blotting, enzyme-linked immunosorbent assays (ELISA) or quantitative immunohistochemistry.

In a preferred embodiment of the invention, antibodies will immunoprecipitate AMPK proteins from solution as well as react with AMPK protein on western or immunoblots of polyacrylamide gels. In another preferred embodiment, antibodies will detect AMPK proteins in paraffin or frozen tissue sections, using immunocytochemical techniques.

Preferred embodiments relating to methods for detecting AMPK include enzyme linked immunosorbent assays (ELISA), radioimmunoassay (RIA), immunoradiometric assays (IRMA) and immunoenzymatic assays (IEMA), including sandwich assays using monoclonal and/or polyclonal antibodies.

Exemplary sandwich assays are described by David et al in U.S. Pat. Nos. 4,376,110 and 4,486,530, hereby incorporated by reference.

Methods for detection also include immuno-fluoresence. Automated and semi-automated image analysis systems may be of use. Several formats for quantitative immunoassays are known. Such systems may incorporate: more than one antibody which binds the antigen; labelled or unlabelled antigen (in addition to any contained in the sample); and a variety of detection systems including radioisotope, colourimetric, fluorimetric, chemiluminescent, and enhanced chemiluminescent; enzyme catalysis may or may not be involved. Immunoassays may be homogenous systems, where no separation of bound and unbound reagents takes place, or heterogeneous systems involving a separation step.

Such assays are commonly referred to as eg enzyme-linked luminescent immunoassays (ELLIA), fluorescence enzyme immunoassay (FEIA), fluorescence immunoassay (FIA), enzyme immunoassay (EIA), luminescent immunoassay (LIA), latex photometrix immunoassay (LPIA).

Methods of cultivating the biological sample (e.g. sample cells) and isolating proteins are well known in the art. Cells can be harvested and lysed and the presence of the protein in the supernatant can be detected using antibodies. Such antibodies are useful in cancer diagnosis. Suitably, the antibodies of the invention are detectably labelled, for example they may be labelled in such a way that they may be directly or indirectly detected. Conveniently, the antibodies may be labelled with a radioactive moiety or a coloured moiety or a fluorescent moiety, or they may be linked to an enzyme. Typically, the enzyme is one which can convert a non-coloured (or non-fluorescent) substrate to a coloured (or fluorescent) product. The antibody may be labelled by biotin (or streptavidin) and then detected indirectly using streptavidin (or biotin) which has been labelled with a radioactive moiety or a coloured moiety or a fluorescent moiety, or the like or they may be linked to an enzyme of the type described above.

As mentioned previously, preferably the cancer is breast cancer or endometrial cancer.

In accordance with an ninth aspect of the invention, there is provided a kit for aiding assessment of a patient's risk of developing cancer, or likely severity or likelihood of progression of cancer, or aiding in selection of a cancer treatment regime for the patient, or aiding in assessment of a cancer treatment regime, the kit comprising a nucleic acid molecule according to the first, second, third and fourth aspects of the invention or molecule according to the fifth aspect of the invention and a package insert containing instructions using the kit.

In accordance with a tenth aspect of the invention, there is provided the use of cells of a lymphoblastoid cell line for studying p53 signalling or in performing a screen for identifying modulators of p53 signalling.

By “modulators”, it is meant to refer to any moiety that modulates the activation, inhibition, delay, repression or interference of one or more of; the activity of p53 signalling.

In accordance with a eleventh aspect of the invention, there is provided a method for studying p53 signalling or for performing a screen for identifying modulators of p53 signalling comprising the step of assessing cells of a lymphoblastoid cell line.

The invention will now be described with reference to the following none limiting figures and examples.

All references herein mentioned are hereby incorporated by reference.

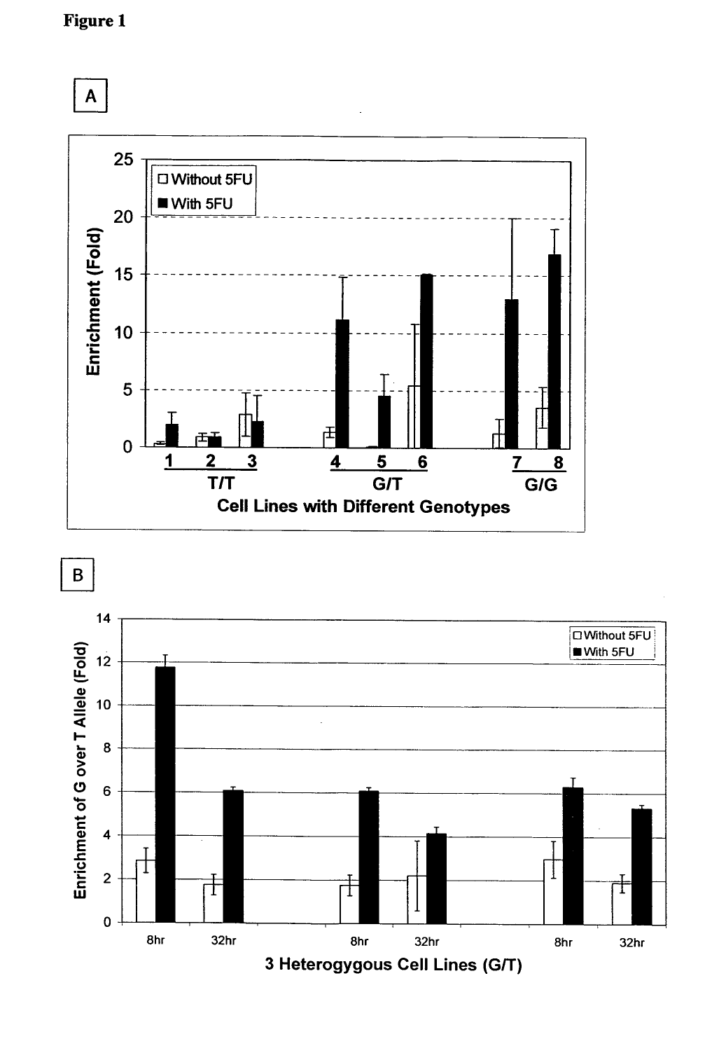

FIG. 1. The results from ChIP and real-time PCR analyses, showing that the wild-type allele (G) is associated with stronger p53 binding activity than the mutant allele (T) in LCLs. A: the differential enrichment of the binding site sequence at the baseline and after 5FU treatment in the cell lines carrying either only wild-type allele (G/G) (two cell lines), or mutant (TM allele (three cell lines), or both alleles (G/T) (three cell lines). B: the enrichment of the wild-type G allele over the mutant T allele in the ChIP pull-down DNAs from the three heterozygous cell lines (G/T) after 5FU treatment for 8 and 32 hours.

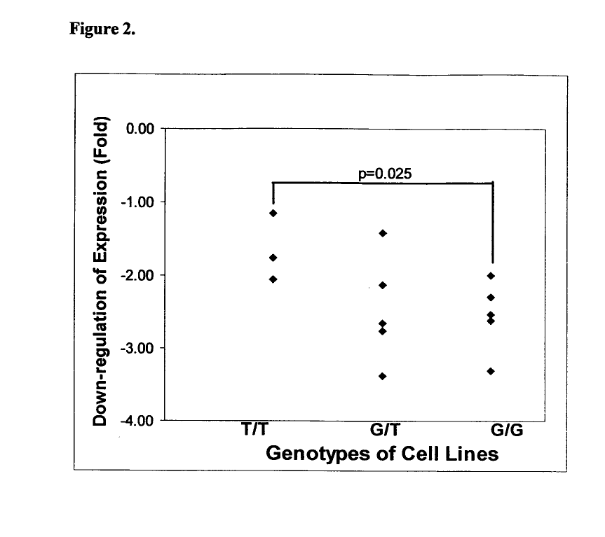

FIG. 2. The results of the real-time gene expression analysis, showing the down-regulation of PRKAG2 expression after 5FU treatment in 13 cell lines carrying either only wild-type allele (G/G) (five cell lines), or mutant (T/T) allele (three cell lines), or both alleles (G/T) (five cell lines).

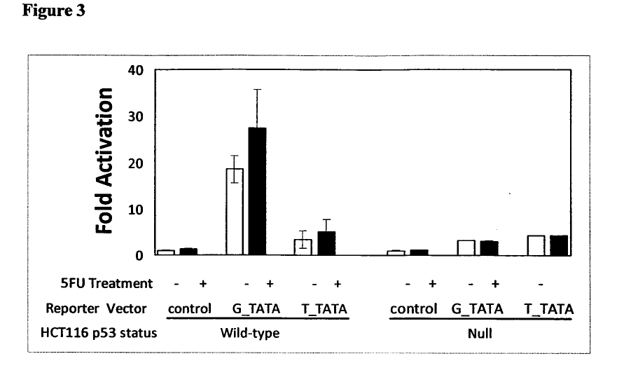

FIG. 3. Functional analysis of the binding site sequence (226 by fragment) and its polymorphism (rs184672) by reporter gene assay in wild-type and p53-null HCT116 cells with or without 5FU treatment. Control: TATA-luciferase pGL4 vector; G_TATA: TATA-luciferase pGL4 vector with a insert of the 226 by binding site sequence of G allele; T_TATA: TATA-luciferase pGL4 vector with a insert of the 226 by binding site sequence of T allele.

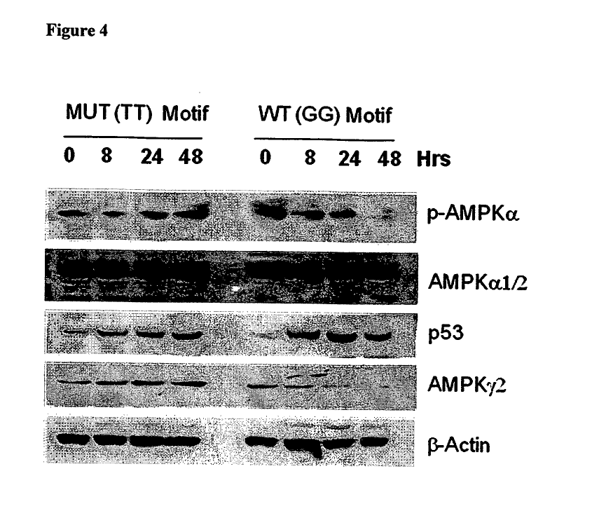

FIG. 4. The results from the western blot analysis, showing the differential down-regulation effect of p53 activation by 5FU on AMPK protein complex (AMPK□, total and phosphorylated-AMPK□proteins) in cells carrying either wild-type (G/G) or mutant (T/T) binding motif.

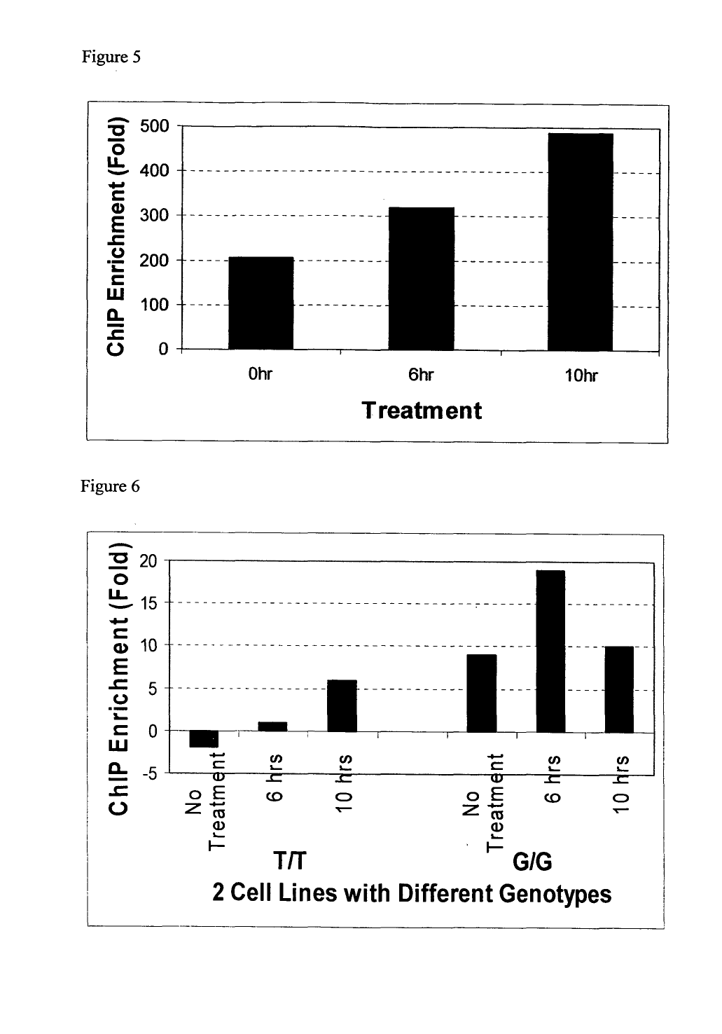

FIG. 5. The results from the ChIP and real-time PCR analysis, showing the significant enrichment of the p53 binding motif sequence of the p21 promoter in the ChIP pull-down DNA from lymphoblastoid cells without or with 5FU treatment for 6 or 10 hrs.

FIG. 6. The results from ChIP and real-time PCR analyses from two cell lines carrying either wild-type (G) or mutant allele (D, showing that the wild-type allele (G) is associated with stronger p53 binding activity than the mutant allele (T) at the baseline (cont) as well as after 5FU treatment for 6 and 10 hrs

TABLE 1
The results of the association analysis in various sample sets under a recessive model of inheritance

| | | | Genotype Freq (%) | | | |
|---|---|---|---|---|---|---|
| Sample Set | Sample | Size | GG or GT | TT | OR (95% CI) | P value |
| Finnish_breast | cases | 2244 | 96.03 | 3.97 | 1.26 (0.85, 1.88) | 0.24 |
| | controls | 1256 | 96.82 | 3.18 | | |
| Swedish_breast | cases | 1297 | 96.14 | 3.86 | 1.45 (0.93, 2.27) | 0.08 |
| | controls | 1484 | 97.3 | 2.7 | | |
| Swedish_endo | cases | 579 | 96.03 | 3.97 | 1.47 (0.83, 2.52) | 0.14 |
| | controls | 1533 | 97.26 | 2.74 | | |
| Combined_breast | cases | 3541 | 96.07 | 3.93 | 1.34 (1.01, 1.77) | 0.04 |
| | controls | 2740 | 97.08 | 2.92 | | |
| Combined_whole | cases | 4113 | 96.06 | 3.94 | 1.36 (1.04, 1.78) | 0.02 |
| | controls | 2938 | 97.11 | 2.89 | | |

Subgroup Analysis in Breast Cancer *

| ER Status | ER+ | 2492 | 96.27 | 3.73 | 1.26 (0.92, 1.72) | 0.15 |
|---|---|---|---|---|---|---|
| | ER− | 588 | 95.58 | 4.42 | 1.48 (0.93, 2.35) | 0.10 |
| Menopause | Post | 2447 | 96.24 | 3.76 | 1.30 (0.96, 1.76) | 0.10 |
| | Pre | 502 | 94.82 | 5.18 | 1.66 (1.00, 2.75) | 0.05 |
| Family History | sporadic | 2359 | 96.31 | 3.69 | 1.25 (0.92, 1.70) | 0.16 |
| | familial | 1098 | 95.72 | 4.28 | 1.48 (1.01, 2.17) | 0.04 |
| | | | Genotypes | | | |
| | | | Frequency (%) | | | |
| Sample Set | Sample | Size | GG or GT | TT | OR (95% CI) | P value |
| Finnish_breast | cases | 2244 | 96.03 | 3.97 | 1.26 (0.85, 1.88) | 0.24 |
| | controls | 1256 | 96.82 | 3.18 | | |
| Swedish_breast | cases | 1297 | 96.14 | 3.86 | 1.45 (0.93, 2.27) | 0.08 |
| | controls | 1484 | 97.30 | 2.70 | | |
| Swedish_endometrial | cases | 579 | 96.03 | 3.97 | 1.47 (0.83, 2.52) | 0.14 |
| | controls | 1533 | 97.26 | 2.74 | | |
| Combined_breast | cases | 3541 | 96.07 | 3.93 | 1.34 (1.01, 1.77) | 0.04 |
| | controls | 2740 | 97.08 | 2.92 | | |
| Combined_whole | cases | 4113 | 96.06 | 3.94 | 1.36 (1.04, 1.78) | 0.02 |
| | controls | 2938 | 97.11 | 2.89 | | |

* All the ORs and p values were calculated by comparing to the combined breast cancer control.

### EXAMPLE 1

Samples: The current Example included the clinical samples from Sweden and Finland. All the Swedish cases were randomly selected from a population-based Swedish cohort that included all Swedish-born breast and endometrial cancer patients between 50 and 74 years of age and resident in Sweden between October 1993 and March 1995. A similar number of age-matched controls were randomly selected from the Swedish Registry of Total Population. All the Swedish cases and controls as well as the source population-based cohort had been described in detail elsewhere (Einarsdottir, Rosenberg et al. 2006) (Einarsdottir, Humphreys et al. 2006; Einarsdottir, Humphreys et al. 2007). Briefly, after informed consent, 1596 breast cancer patients, 719 endometrial cancer patients and 1730 healthy volunteers participated into this study by providing either whole blood or non-malignant paraffin-embedded tissues for DNA analysis. From whole blood samples, DNAs were extracted by using the QIAamp DNA Blood Maxi Kit (Qiagen) according to the manufacturer's instruction. From non-malignant paraffin-embedded tissues, DNA was extracted using a standard phenol/chloroform/isoamyl alcohol protocol (Isola, DeVries et al. 1994).

The Finnish breast cancer cases consist of two series of unselected breast cancer patients and additional familial cases ascertained at the Helsinki University Central Hospital. The first unselected series of 884 breast cancer patients studied were collected at the Department of Oncology, Helsinki University Central Hospital in 1997-1998 and 2000 and cover 79% of all consecutive, newly diagnosed breast cancer cases during the collection periods (Syrjakoski, Vahteristo et al. 2000; Kilpivaara, Bartkova et al. 2005). 876 patients (99%) from this series were successfully genotyped in this Example.

The second unselected series, containing 986 consecutive newly diagnosed breast cancer patients, were collected at the Helsinki University Central Hospital 2001-2004 and covers 87% of all such patients treated at the Department of Surgery during the collection period. Of this series 979 patients (99%) were successfully genotyped.

The series of 538 additional familial breast cancer cases in this study have been collected at the Helsinki University Central Hospital as described (Eerola, Blomqvist et al. 2000). The genotyped series included 295 patients with strong family history, defined as three or more breast or ovarian cancer cases in the first or second degree family members including the index case. These families were screened negative for BRCA1/2 mutations as previously described in detail (Vehmanen, Friedman et al. 1997; Vahteristo, Eerola et al. 2001; Vahteristo, Bartkova et al. 2002). The remaining 243 genotyped familial cases had a single affected first degree family member, for 213 of these cases, the Finnish BRCA1/2 founder mutations have been excluded as described (Vahteristo, Eerola et al. 2001; Vahteristo, Bartkova et al. 2002). All the cancer diagnoses have been verified through the Finnish Cancer Registry and hospital records. Allele and genotype frequencies in the normal population were determined in 1256 healthy female population controls collected from the same geographical region.

SNP Genotyping: genotyping analysis of SNPs was performed by using the MALDI-TOF mass spectrometry-based MassARRAY™ system from the Sequenom (San Diego, Calif., US) (Swedish samples) as well as the TaqMan assays from the AppliedBiosystesm (ABI) (Foster City, Calif., US) (Finnish samples). All genotyping plates included positive and negative controls, DNA samples were randomly assigned to the plates, and all genotyping results were generated and checked by laboratory staff unaware of case-control status.

Lymphoblatoid cell lines and culture: All lymphoblastoid cell lines (LCLs) used in this study were obtained from the Coriell depository (http://www.coriell.org/). Cells were cultured in RPMI medium supplemented with 20% fetal bovine serum. For ChIP, real-time qPCR and western blot analyses, cells were treated with 5FU at the concentration of 375 uM for various hours. All the drug treatments were done during the log phase of cell growth (about 1 to 1.5 millions of cells per ml). Cells were harvested after culture with or without drug treatment(s) and stored at −80 degrees. 5FU was obtained from the Sigma.

ChIP Analysis: ChIP assays were performed in LCLs using the protocol described previously (Weinmann and Farnham 2002; Wells and Farnham 2002). For all ChIP analyses, the DO1 monoclonal antibody for p53 (Santa Crux Biotechnology, Santa Cruz, Calif.) was used for immunoprecipitation, and real-time quantitative PCR analyses were performed using the PRISM 7900 Sequence Detection System and the SYBR protocol as described (Ng et at 2003).

The real-time PCR analysis was performed using the following primers:

(For PRKAG2)
CCATCCTGCCTGAGCATGTCTGAAC (forward)
and
CCGGCTTTGCCAGACAATTGG (reverse);
(For p21)
CAGGCTGTGGCTCTGATTGGCTTTC (forward)
and
GCTGGCAGATCACATACCCTGTTCAGAGTA (reverse);
(For Actin)
ACCCACACTGTGCCCATCTACGAG (forward)
and
TCTCCTTAATGTCACGCACGATTTCC (reverse).

The primers were designed using Vector NTI. Relative occupancy was calculated by determining the immunoprecipitation efficiency (ratios of the amount of immunoprecipitated DNA over that of the input sample) and normalized to the level observed at a control region, which was defined as 1.0. The control region was a distal site around the binding site for Actin and not enriched by the immunoprecipitation. Each real-time quantitative PCR analysis was done in triplicate.

Allele Enrichment Analysis of ChM pull-down DNAs by real-time PCR: the allele enrichment analysis of the ChIP input and pull-down DNAs from heterozygous cell lines was performed by real-time quantitative PCR using a made-to-order TaqMan SNP assay for rs1804674 from the ABI. The quality of the TaqMan SNP assay was first verified by genotyping 30 CEPH DNA samples, and all the genotype results are consistent with the ones from the HapMap project (data not shown). For real-time PCR analysis, the Ct value difference (ΔCt) between G and T alleles of a ChIP pull-down DNA was normalized by the ΔCt value of the corresponding input DNA (reflecting the equal numbers of G and T alleles in normal genomic DNAs from the heterozygous cell lines). The normalized ΔCt value (ΔΔCt) was then used to calculate the enrichments (Fold Change using the formula of 2^{ΔΔCt}) of the wild-type G allele over the mutant T allele in the ChIP pull-down DNA. All the real-time PCR analyses were done in triplicate.

Expression Analysis by Real-time PCR: total RNAs were extracted from cells (with or without 5FU treatment) using the RNeasy Kit from the Qiagen (with DNase digestion step). 200 ng RNA was then reverse transcribed into 20 μl cDNA using the SuperScript kit from the Invitrogen (CA, USA), and real-time PCR analysis was subsequently performed by using 2 μl cDNA as template. All the real-time PCR analyses were done in the ABI Prism 7700 sequence detection system by using the TaqMan assays from the ABI. For PRKAG2, assay-by-demand assay was developed by using the Primer Express software from the ABI: GTTTCCCCTGGAATCCTATAAGC (Forward), CGAGGCATAGATGCGATTCTC (reverse) and CGAGCCTGAACGGT (probe). For normalization, a ready-to-use TaqMan probe for the glyceraldehyde-3-phosphate dehydrogenase (GAPDH) gene was analyzed as endogenous control. Each real-time PCR analysis was done in triplicate.

All the Ct values from the real-time PCR analyses were analyzed by using the comparative Ct method provided by the manufacturer (ABI). Briefly, the Ct values from the PRKAG2 analysis were first normalized by the Ct values of the endogenous control, GHAP. The normalized Ct (ΔCt) values were then used to calculate the Ct value difference (ΔΔCt) between 10 h treatment and the baseline. Fold change in the expression of PRKAG2 between the baseline and the 10h treatment of 5FU was calculated by using the formula of 2^{ΔΔCt}.

Promoter Assay Analysis: A 226 by region encompassing the intronic p53 binding site within PRKAG2 was amplified using hotstart PCR with forward primer 5′-TAGGAGACCTGGGGGACTTT-3′ and reverse primer 5′-CAGGCATCTCGAAGAGATCA-3′ and 50 ng of genomic DNAs isolated from the individuals carrying either the wild-type (WT) G or mutant (MUT) A allele. The PCR conditions were; 94° C. for 15 mins, followed by 35 cycles of denaturation at 94° C. for 45 s, annealing 55° C. for 45 s, and extension at 72° C. for 45 s. The resultant PCR products of 226 by were purified from agarose gels and cloned using TOPO-TA cloning system (Invitrogen, Calsbad, Calif.). The genotypes of the cloned DNA fragments were confirmed by DNA sequencing. Subsequently, the DNA fragments were subcloned into the upstream of TATA-luciferase (fire-fly) containing pGL4 vector (Promega) using Kpn I and Xho I restriction enzymes (New England Biolabs).

Reporter assay analysis was performed by using both HCT116 wild type and null for p53 cells (provided by Dr Bert Vogelstein's lab at the Johns Hopkins School of Medicine) that were maintained in DMEM containing 10% fetal bovine serum. 5×10⁴ cells were plated in triplicate in 24-well plates and transfected next day with 250 ng of either parent TATA-luc, WT-TATA-luc or MUT-TATA-luc plasmid DNAs under serum free conditions using 1 μg per well of Lipofectamine 2000 (Invitrogen, Calsbad, Calif.). 2.5 ng of pRL-CMV vector containing renilla luciferase was co-transfected in each well to normalize transfection efficiency across wells. After 8 hours the cells were recovered for 3 hours in serum containing medium, following which the cells were treated for 12 hours with 375 μM 5-Fluorouracil or DMSO. The cells were lysed in passive lysis buffer and promoter assays were carried out as per manufacturer's instructions using Promega Dual-luciferase assay system. The values obtained for each construct were normalized as fold-change to that of the activity of parental TATA-luc vector in HCT116 WT cells (designated as 1).

Western Blot Analysis: Total protein was extracted from cells using the Modified RIPA buffer. The Micro BCA Protein Assay Reagent Kit (Pierce, Rockford, Ill., U.S.A) was used to quantify protein concentration. Western blot was performed using 40 μg of protein using the established protocol and the following antibodies: 1) antibody for actin (control, 1:5000 dilution), 2) p53(DO-1) sc-126 (Santa Crux Biotechnology, 1:1000 dilution); 3) AMPKα, Phospho-AMPKα (Thr172) antibodies for both the total- and phosphor-AMPK proteins (Cell Signaling technology, 1:1000 dilution), and 4) AMPKγ2 antibody (Cell Signaling technology, 1:1000 dilution).

Statistical Analysis: Hardy-Weinberg Equilibrium (HWE) test was performed in the Finnish and Swedish control samples separately, and no evidence for deviation from HWE was found. Association analysis was performed using the X² test under a recessive model of inheritance. For the joint association analyses of the combined Swedish-Finnish breast cancer sample and the combined breast-endometrial cancer sample, the Mantel-Haenszel method for meta-analysis was used by assuming fixed effect. For the joint analysis of the breast-endometrial sample, the Swedish cases were defined as having either breast or endometrial cancer. All statistical analyses were performed by using the StataSE8 system.

Results

Identification of p53 Binding Site SNPs

Of 542 high confidence p53 binding sites identified in HCT116 cell line by our genome-wide ChIP-PET mapping analysis (Wei, Wu et al. 2006), 235 sites were selected for SNP mining where an unequivocal p53 consensus binding motif sequence (5′-RRRCINWGYYYRRRCWWGYYY-3, can be found. The sequences of the 235 binding sites were blasted against the dbSNP database (version 115), and 14 SNPs were identified to be directly located within the binding motifs. Of the 14 SNPs, 12 SNPs were successfully genotyped in 76 anonymous germ-line DNA samples of Caucasian population, and 6 SNPs were confirmed to be polymorphic with a minor allele frequency (MAF) above 1%.

Of the 6 confirmed p53 binding motif germ-line polymorphisms, rs1860746 was found to be located within the consensus motif sequence of an p53 binding site in the third intron of the PRKAG2 gene where high p53 protein occupancy was observed (Wei, Wu et al. 2006). rs1860746 (a G/T substitution) is located at one of the highly conserved bases of p53 motif sequence, and its minor allele T causes a mismatch to the p53 consensus motif sequence: 5′-RRRCWWGYYYRRRCWW[G/T]YYY-3′. The binding site carrying the major allele G has a perfect p53 consensus motif sequence and is therefore expected to be associated with good p53 binding, whereas the site carrying the minor allele T has a mismatch to the p53 consensus motif sequence and is thus we postulate to be associated with weaker p53 protein binding. The genotyping analysis of the SNP in 76 CEPH germ-line DNA samples revealed its MAF to be 20%, which is consistent with the result from the HapMap project. Interestingly, according to the results from the HapMap project, the MAF of this SNP in Asian populations (Chinese and Japanese) is only about 1%, as compared to the higher MAF of 20% observed in African and Caucasian populations.

That PRKAG2 encodes the gamma 2 noncatalytic subunit of the AMPK protein complex, a central sensor of energy stress, suggests that this germ-line p53 binding motif SNP may act as a cis-regulatory variant linking p53 and metabolic homeostasis. This, coupled with the known involvement of AMPK and p53 in cancer development and its interesting frequency pattern in different population, encouraged us to characterize the molecular and physiological function of this germ-line p53 binding motif polymorphism in cancer development.

Molecular Characterization of the p53 Binding Site Within PRKAG2 and Its Germ-Line Polymorphism (rs1860746)

To characterize the molecular function of this p53 binding site and its germ-line binding motif polymorphism, we chose lymphoblastoid cell lines (LCLs) as in-vitro system because LCLs have a normal diploid genome and a large collection of cell lines where cells carrying different genotypes of germ-line SNPs are available for functional analysis. We performed ChIP analysis in LCLs by using a confirmed p53 binding site sequence within the promoter region of the well-characterized p53 target gene CDKNIA(p21) (Kaeser and Iggo 2002) and found that there is a significant enrichment of the p53 binding motif sequence of the p21 promoter in the ChIP pull-down DNA from LCL at the baseline (about 200 fold enrichment) and after the activation of the p53 protein by 5-fluorouracil (5FU) treatment for 6 (about 300 fold higher) and 10 hrs (about 500 fold higher) (see FIG. 5). Western blot analysis (see FIG. 4) also showed that the expression of p53 protein in LCLs can be induced in a time-dependent fashion by 5FU treatment. LCL is therefore a good diploid cellular system for studying p53-mediated cellular response.

To investigate rs1860746's impact on p53's binding to its intronic binding site within PRKAG2, the ChIP analysis was performed in 8 LCL cell lines: three homozygous for the mutant T allele; two homozygous for the wild-type G allele, and three heterozygous. A significant enrichment of the binding site sequence was observed at the baseline and further augmented after 5FU treatment (for 10 hrs) in the five cell lines that carry either one or two copies of the wild-type G allele (12 fold enrichment in average), whereas the three cell lines carrying two copies of the mutant T allele showed little enrichment of binding sequence (FIG. 1A) (2 fold enrichment in average). To further demonstrate the stronger binding of p53 to the wild-type binding motif (G allele) than the mutant binding one (T allele), the relative abundances of the wild-type (G allele) and mutant (T allele) motif sequences in the ChIP pull-down DNAs from the three heterozygous cell lines after 5FU treatment were directly measured by real-time PCR analysis. As demonstrated in FIG. 1B, after 5FU treatment for 6 or 32 hours, significantly more of the wild-type G allele sequences than the mutant T allele sequences were found in the ChIP pull-down DNAs (5 to 10 fold enrichment of wild-type over mutant alleles). The enrichment of the wild-type G over mutant T allele could also be observed at the baseline, although the enrichment is less prominent. The series of the ChIP analyses clearly show that the p53 protein has a higher binding affinity to the wild-type G allele than to the mutant T allele, although the single base substitution does not totally abolish p53's binding to this site.

To investigate whether the observed differential binding activity will lead to the difference in the expression activity of its putative target gene PRKAG2, the transcription of PRKAG2 mRNA (with or without 5FU treatment) using real-time quantitative PCR (qPCR) in13 cell lines with different genotypes were first analysed: three cell lines homozygous for the mutant T allele, five cell lines homozygous for the wild-type G allele, and five heterozygous cell lines (G/T). In most of the cell lines, there is a down-regulation of PRKAG2 expression after 5FU treatment (FIG. 2). Furthermore, the down-regulation of PRKAG2 expression in the five homozygous cell lines for the wild-type G allele is significantly stronger that the down-regulation in the three homozygous cell lines for the mutant T allele (p=0.025, t-test). Interestingly, the difference of p53 binding activity and the down-regulation of its target gene expression was observed only in the cells carrying two copies of the mutant motif (T allele) when compared to those homozygous for the wild-type configuration. To further investigate whether the suppressive transcriptional regulation is p53 dependent, the transcription regulatory activities of the wild-type and mutant binding site sequences were directly measured through a reporter assay analysis. Both wild-type and mutant binding site sequences were cloned into a TATA-luciferase reporter vector and then transfected into HCT116 cells with either wild-type p53 protein or with the p53 disrupted by homologous recombination (p53 null). In the p53 wild-type HCT116 cells, the presence of the wild-type binding site sequence can strongly induce the expression of the reporter gene (20 fold induction), and the induction is augmented by the activation of p53 by 5FU treatment (about 30 fold induction) (FIG. 3). In the p53 null HCT116 cells, this induction effect by the wild-type binding site sequence was largely abolished. In both p53 wild-type and null HCT116 cells, the mutant binding site sequence (T allele) shows a minimal induction of the report gene expression. This result provides direct evidence for this binding site sequence to be associated with a p53-dependent transcriptional regulatory activity. Interestingly, both ChIP and real-time gene expression analyses indicate that the transcriptional impact of this p53 motif polymorphism is largely restricted to the cells carrying only mutated p53 motif. Furthermore, large difference in the expression activity can be observed across the cell lines carrying the same genotype of the p53 motif polymorphism, suggesting that the expression activity is also influenced by other factors.

AMPK protein complex consists of one catalytic (a) and two non-catalytic regulatory (β and γ) subunits, and the expression and activity of the AMPK protein complex depends on the co-regulation of its three subunits¹²⁻¹⁴. This raises the possibility that by interrupting p53′s down-regulation effect on the transcriptional expression of the AMPKγ subunit, this germ-line p53 binding motif variant can have an impact on the expression and activity of the AMPK protein complex. To verify this possibility, we investigated the polymorphism's impact on the protein levels of both AMPK γ and a subunits using western blot analysis. The western blot analysis was performed in two cell lines (among the 13 cell lines subjected to real-time PCR analysis) that show the most prominent difference in the p53-mediated down-regulation of PRKAG2 mRNA level. Protein levels of p53, total and phosphorylated AMPKγ, AMPKα and actin (endogenous control) were assessed in the two cell lines at baseline and after 5FU treatment for 8, 24 and 48 hours. As shown in FIG. 4, the expression of p53 protein was induced in a time-dependent fashion by 5FU treatment in both cell lines. In contrast the levels of the AMPK□ and total and phosphorylated-AMP□proteins after 5FU treatments differ significantly between the two cell lines. In the cell line carrying mutant binding site (T/T), the levels of the AMPKγ and total and phosphorylated-AMPKα proteins were largely unaffected by 5FU treatment, whereas in the cell line carrying wild-type binding site (G/G), a significantly decreased expression of the three proteins was seen after 5FU treatment, primarily of phosphorylated AMPKα and AMPKγ especially at 48 hours. These results are consistent with the regulatory effect of AMPKγ on the activity of AMPKα.

Genetic Association Analysis of the p53 Binding Motif SNP (rs180746) in Breast and Endometrial Cancer Susceptibility

Given that both AMPK and p53 have been implicated in cancer development^{15,16}, this germ-line regulatory variant (rs180746) of the transcriptional link between p53 and AMPK may have an impact on cancer susceptibility. To test this hypothesis, the SNP rs180746 in three well characterized patient samples of breast and endometrial cancers from Sweden and Finland were analysed. The “worst case” assumption is that the effect of this SNP on cancer susceptibility will be low, as has been found for the recent identified breast cancer susceptibility loci¹⁷, and that only the homozygous TT genotype would show a phenotypic effect (as indicated by our in vitro functional analyses). We genotyped the SNP in 1297 breast cancer patients, 579 endometrial cancer patients and 1637 healthy controls from Sweden and 2399 breast cancer patients and 1256 healthy controls from Finland. The MAF of rs1860746 is 18.3% and 18.1% in the Swedish and Finnish samples respectively, which is very similar to the one detected in the 76 CEPH DNA samples as well as the one reported in Caucasians by the HapMap project. The genotype frequencies at this locus are in Hardy-Weinberger equilibrium in both Swedish and Finnish samples.

The role of the rs1860746 in breast cancer susceptibility was examined. As shown in the Table 1, the homozygous mutant genotype (TT) is associated with an increased risk for breast cancer in either the Swedish or the Finnish samples (Odds Ratios 1.45 and 1.26 respectively), though there was a trend, the evidence achieved statistical significance in neither sample, likely due to the low frequency of the homogenous mutant genotypes in each sample (˜3%). To improve the statistical power for detection, a joint analysis of the Swedish and Finnish breast cancer samples were performed. When the two studies were combined, the association of the TT genotype with breast cancer risk achieved significance (OR=1.34, p=0.043) (Table 1). The role of this regulatory polymorphism in different clinical patients stratified based on the menopausal, family history and ER status were investigated. Interestingly, it was observed that the significant genetic evidence in the premenopausal patients (OR=1.66, p=0.05) and the patients with family history (OR=1.48, p=0.04). No significant evidence was observed in either sporadic (OR=1.25, p=0.16) or postmenopausal (OR=1.38, p=0.10) cases. Significant evidence in the subgroup analysis based on ER status was also not observed. It was then tested whether the TT genotype could also confer increased risk in another related cancer, endometrial cancer. With only 579 cases, a trend towards an increased risk was found: OR=1.47 (P=0.143). Finally, a joint analysis was performed of all the three cancer samples, which sustained the significant association between the homozygous mutant (T/T) genotype and cancer susceptibility (Table 1): OR=1.36, p=0.024.

Discussion

By carrying out a series of functional analyses in LCL cells, it was discovered the γ2 subunit of AMPK protein complex (coded by PRKAG2) to be a downstream target of p53's transcriptional regulation. It is shown that a chain of events occurs in the LCL cells carrying the wild-type p53 binding motif within PRKAG2 following exposure to a genotoxic agent: the activation of p53 protein (e.g., by 5FU treatment) increases the binding of p53 to the binding site, which in turn down-regulates the expression of AMPKγ (PRKAG2) (and as a result, the a subunit as well). When this binding site is interrupted by germ-line variant (rs1860746), p53 binding activity at this binding site is significantly reduced, which in turn results in an attenuated down-regulation of AMPK expression. This suggests that p53 is a suppressor in of AMPK expression and that there is variation in human populations in this interaction at the molecular level.

AMPK is known as the central sensor of energy stress and regulated by the AMP/ATP ratio. When cells face cellular stress such as glucose deprivation, the AMP/ATP ratio is elevated, and as a consequence, AMPK protein is activated. Activation of AMPK protein inhibits energy-consuming processes such as protein synthesis and promotes energy-generating processes such as glucose uptake and fatty acid metabolism, allowing cells to restore energy balance and thus survive cellular stress¹⁸. p53 has been shown to be a down-stream target of AMPK¹⁹. Activation of AMPK protein can enhance the activity of p53 by stabilizing the p53 protein through phosphorylation of its Ser-15 residue. This discovery of the down-regulation effect of p53 on the expression of AMPK protein suggests a novel negative feed-back impact of p53 on AMPK that further complicates the regulatory dynamics of these two genes and their gene products. Intriguingly, activating mutations in the human PRKAG2 gene is associated with a cardiac glycogen storage disorder associated with ventricular preexcitation arrhythmias (Wolff-Parkinson-White syndrome)^{20,21}. This raises the possibility that rs1860746 homozygotes, which is associated with a conditional up-regulation of AMPKγ activity, may also exhibit cardiac phenotypes under myocardial stress.

This Example also provides new evidence for the emerging role of p53 in regulating energy homeostasis. P53 is the best known as a tumor suppressor gene and is mechanistically involved in genome surveillance¹⁵. When activated in response to stress signals, p53 can trigger multiple cellular processes including cell-cycle arrest, senescence and apoptosis. Recent data have shown that p53 plays a broader role than as the tumor suppressor gene and might be involved in other biological processes such as metabolism but the molecular mechanisms of this involvement are not well understood²². p53 has been shown to down-regulate the expression of the phosphoglycerate mutase (PGM) whose over-expression can enhance glycolysis and bypass senescence²³. Moreover, the loss of p53 activity can lead to a reduction of oxidative respiration and an enhancement of aerobic glycolysis by transcriptionally down-regulating SCO2 protein²⁴. More recently, it was shown that the activation of p53 can directly inhibit glycolysis and stimulate oxidative respiration through the transcriptional activation of the TIGAR gene²⁵. By identifying p53 to be an up-stream regulator of the AMPK protein, this Example suggests a new mechanism for p53 to regulate energy homeostasis. Interestingly, this Example also found p53 to behave as a suppressor to regulate the expression of AMPK protein, which is consistent with the emerging discovery of p53 acting as a transcriptional suppressor (reference).

AMPK plays an important role in tumor development, but through a complex and partially understood mechanism. On one hand, several lines of experimental results suggested a ‘tumor suppressor’ function of AMPK. The identification of LKB1, a tumor suppressor gene, as the direct upstream regulator of AMPK provided a first link between AMPK and the regulation of tumor cell growth²⁶. This link was further enhanced by the subsequent identification of two more tumor suppressor genes, p53 and TSC2, as the direct downstream targets of AMPK^{19,27}. On the other hand, AMPK can also promote tumor cell growth by facilitating the ‘metabolic switch’ from oxidative respiration to glycolysis, one of the hallmarks for cancer development. It has been shown that AMPK expression can promote tumor development by increasing tumor cells' tolerance to nutrient starvation²⁹. A more recent study has shown that in mouse xenograft model, the absence of AMPK activity greatly inhibits tumor growth²⁹. These results indicate that AMPK can promote tumor growth by increasing the adaptation of tumor cells to the hypoxic and glucose-deprived microenvironments common in solid tumors. Taken together, the in-vitro molecular data revealed a rather complex and confusing dynamics of AMPK function as both tumor suppressor and promoter.

Underscoring this complexity, a recent study has demonstrated an activating effect of p53 on the expression of AMPKβ1 subunit, but without impact on the expression of the α subunit³⁰. By contrast, results of the present Example observed the repression of AMPKγ and by p53. The and y subunits are regulatory subunits to AMPKα but are distinct Feng et al investigated only the short term impact of p53 activation (less than 24 hrs) on the AMPKα total protein and found no change in the levels. In this Example, the suppressive effect of p53 at the earlier time points was found only in the phosphorylated AMPKα protein. The suppressive effect on the total α protein level only emerged after prolonged treatment of 5FU for 48 hrs. Therefore, there may be no substantive difference between the studies concerning the enzymatic α subunit. Given the differences in experimentation both in design and the cellular substrates used (epithelial cancer cell lines vs. lymphoblastoid cell lines), further clarification will be needed.

This Example provides the first genetic evidence, at the population level, that conditionally higher AMPK activity is associated with increased susceptibility to cancer development. By genotyping this p53 binding site polymorphism in three cancer sample sets, consistent evidence for association of the homozygous mutant genotype of the polymorphism with high susceptibility to cancer development was found (OR=1.36, p=0.024, based on 4113 cancer cases and 2938 healthy controls). Because the mutant allele is shown to interrupt p53′s down-regulation of the PRKAG2/AMPK expression under conditions of genotoxic stress, our genetic analysis strongly suggests that AMPK more likely functions as a tumor promoter, at least at human population level.

Interestingly, the subgroup analysis in breast cancer suggested that this germ-line p53 binding site polymorphism may play a more prominent role in pre-menopausal breast cancer patients with a positive family history. This finding is consistent with the observations that patients with germ-line p53 mutations in the families affected with Li-Fraumeni syndromes (LFS) are at risk for early-onset breast cancer³¹ and that germ-line p53 mutations can be found in the patients of hereditary breast cancer who are negative for BRAC1 and BRAC2 mutations³². Furthermore, the association of this germ-line regulatory variant with endometrial cancer points to the notion that the genetic effect might play a role in the development of other cancers as well. Given that there is good evidence for p53 and metabolic stress to function in the aging process³³, it appears that that the germ-line p53 binding site polymorphism may also have an impact on some aspect of human longevity.

This Example presents one of the few efforts where p53-related regulatory variants were investigated molecularly and genetically³⁴. The most intensively studied p53-related regulatory variant is the T/G polymorphism within the intronic promoter of MDM2 that was shown to influence the MDM2 expression by modulating Sp1 binding to MDM2 and therefore be associated with decreased level of p53 protein and accelerated tumor formation in humans⁵. Mendendez et al identified a C-to-T polymorphism within the proximal promoter region of the flt-1gene, where the minor allele of T created a half-binding site for p53 and brought the system under the control of p53 network³⁵. A more recent effort by the same group has further demonstrated that the presence of this polymorphism also created a partial responsible element for estrogen receptor upstream the previous identified half-binding site for p53, which introduces a mechanism for synergistic simulation of transcription at this flt-promoter site through the combined action of p53 and ER³⁵. The importance of these p53-related regulatory variants in disease development, however, has not been demonstrated.

In summary, the present invention shows that p53 to be an up-stream regulator of the AMPK protein through an intronic p53 binding site within the AMPKγ subunit gene and provided evidence for the modulation of this transcriptional linkage between p53 and AMPK by a germ-line binding motif polymorphism. More importantly, the present invention has further demonstrated that this modulation of p53-AMPK transcriptional link by the germ-line polymorphism will increase the risk for cancer development. As an proof-in-principal study, this invention has highlighted that combining the genome-wide discovery of transcription regulatory elements (such as transcription factor binding sites) with the forward genetic analysis in both model and human systems can greatly advance our understanding on the molecular and physiological functions of regulatory genetic variation. A ‘marriage’ between the new genome-wide knowledge of various regulatory sequences and the rapidly accumulated disease association data on germ-line polymorphisms will bring a paradigm shift to regulatory variation research.

### References

1. Wray, G. A. The evolutionary significance of cis-regulatory mutations. Nat Rev Genet 8, 206-16 (2007).
2. King, M. C. & Wilson, A. C. Evolution at two levels in humans and chimpanzees. Science 188, 107-16 (1975).
3. Knight, J. C. Regulatory polymorphisms underlying complex disease traits. J Mol Med 83, 97-109 (2005).
4. Sladek, R. & Hudson, T. J. Elucidating cis- and trans-regulatory variation using genetical genomics. Trends Genet 22, 245-50 (2006).
5. Bond, G. L. et al. A single nucleotide polymorphism in the MDM2 promoter attenuates the p53 tumor suppressor pathway and accelerates tumor formation in humans. Cell 119, 591-602 (2004).
6. Hu, Z. et al. MDM2 promoter polymorphism SNP309 contributes to tumor susceptibility: evidence from 21 case-control studies. Cancer Epidemiol Biomarkers Prev 16, 2717-23 (2007).
7. Bourque, G. et al. Evolution of the mammalian transcription factor binding repertoire via transposable elements. Genome Res (2008).
8. Pastinen, T. & Hudson, T.J. Cis-acting regulatory variation in the human genome. Science 306, 647-50 (2004).
9. Cawley, S. et al. Unbiased mapping of transcription factor binding sites along human chromosomes 21 and 22 points to widespread regulation of noncoding RNAs. Cell 116, 499-509 (2004).
10. Wei, C. L. et al. A global map of p53 transcription-factor binding sites in the human genome. Cell 124, 207-19 (2006).
11. Kaeser, M. D. & Iggo, R. D. Chromatin immunoprecipitation analysis fails to support the latency model for regulation of p53 DNA binding activity in vivo. Proc Natl Acad Sci USA 99, 95-100 (2002).
12. Crute, B. E., Seefeld, K., Gamble, J., Kemp, B. E. & Witters, L. A. Functional domains of the alpha1 catalytic subunit of the AMP-activated protein kinase. J Biol Chem 273, 35347-54 (1998).
13. Dyck, J. R. et al. Regulation of 5′-AMP-activated protein kinase activity by the noncatalytic beta and gamma subunits. J Biol Chem 271, 17798-803 (1996).
14. Elbing, K., Rubenstein, E. M., McCartney, R. R. & Schmidt, M. C. Subunits of the Snf1 kinase heterotrimer show interdependence for association and activity. J Biol Chem 281, 26170-80 (2006).
15. Vousden, K. H. & Lu, X. Live or let die: the cell's response to p53. Nat Rev Cancer 2, 594-604 (2002).
16. Shaw, R. J. Glucose metabolism and cancer. Curr Opin Cell Biol 18, 598-608 (2006).
17. Easton, D. F. et at. Genome-wide association study identifies novel breast cancer susceptibility loci. Nature 447, 1087-93 (2007).
18. Motoshima, H., Goldstein, B. J., Igata, M. & Araki, E. AMPK and cell proliferation-AMPK as a therapeutic target for atherosclerosis and cancer. J Physiol 574, 63-71 (2006).
19. Jones, R. G. et al. AMP-activated protein kinase induces a p53-dependent metabolic checkpoint. Mol Cell 18, 283-93 (2005).
20. Arad, M. et al. Constitutively active AMP kinase mutations cause glycogen storage disease mimicking hypertrophic cardiomyopathy. J Clin Invest 109, 357-62 (2002).
21. Arad, M. et al. Glycogen storage diseases presenting as hypertrophic cardiomyopathy. N Engl J Med 352, 362-72 (2005).
22. Vousden, K. H. & Lane, D. P. p53 in health and disease. Nat Rev Mol Cell Biol 8, 275-83 (2007).
23. Kondoh, H. et al. Glycolytic enzymes can modulate cellular life span. Cancer Res 65, 177-85 (2005).
24. Matoba, S. et al. p53 regulates mitochondrial respiration. Science 312, 1650-3 (2006).
25. Bensaad, K. et al. TIGAR, a p53-inducible regulator of glycolysis and apoptosis. Cell 126, 107-20 (2006).
26. Shaw, R. J. et al. The tumor suppressor LKB1 kinase directly activates AMP-activated kinase and regulates apoptosis in response to energy stress. Proc Natl Acad Sci USA 101, 3329-35 (2004).
27. Inoki, K., Zhu, T. & Guan, K. L. TSC2 mediates cellular energy response to control cell growth and survival. Cell 115, 577-90 (2003).
28. Kato, K. et al. Critical roles of AMP-activated protein kinase in constitutive tolerance of cancer cells to nutrient deprivation and tumor formation. Oncogene 21, 6082-90 (2002).
29. Laderoute, K. R. et al. 5′-AMP-activated protein kinase (AMPK) is induced by low-oxygen and glucose deprivation conditions found in solid-tumor microenvironments. Mol Cell Biol 26, 5336-47 (2006).
30. Feng, Z. et al. The regulation of AMPK beta1, TSC2, and PTEN expression by p53: stress, cell and tissue specificity, and the role of these gene products in modulating the IGF-1-AKT-mTOR pathways. Cancer Res 67, 3043-53 (2007).
31. Olivier, M. et al. Li-Fraumeni and related syndromes: correlation between tumor type, family structure, and TP53 genotype. Cancer Res 63, 6643-50 (2003).
32. Walsh, T. et al. Spectrum of mutations in BRCA1, BRCA2, CHEK2, and TP53 in families at high risk of breast cancer. JAMA 295, 1379-88 (2006).
33. Bensaad, K. & Vousden, K. H. p53: new roles in metabolism. Trends Cell Biol 17, 286-91 (2007).
34. Pietsch, E. G., Humbey, O. & Murphy, M. E. Polymorphisms in the p53 pathway. Oncogene 25, 1602-11 (2006).
35. Menendez, D. et al. A single-nucleotide polymorphism in a half-binding site creates p53 and estrogen receptor control of vascular endothelial growth factor receptor 1. Mol Cell Biol 27, 2590-600 (2007).
36. Einarsdottir, K. et al. Comprehensive analysis of the ATM, CHEK2 and ERBB2 genes in relation to breast tumour characteristics and survival: a population-based case-control and follow-up study. Breast Cancer Res 8, R67 (2006).
37. Einarsdottir, K. et al. Linkage disequilibrium mapping of CHEK2: common variation and breast cancer risk. PLoS Med 3, e168 (2006).
38. Einarsdottir, K. et al. Effect of ATM, CHEK2 and ERBB2 TAGSNPs and haplotypes on endometrial cancer risk. Hum Mol Genet 16, 154-64 (2007).
39. Isola, J., DeVries, S., Chu, L., Ghazvini, S. & Waldman, F. Analysis of changes in DNA sequence copy number by comparative genomic hybridization in archival paraffin-embedded tumor samples. Am J Pathol 145, 1301-8 (1994).
40. Syrjakoski, K. et al. Population-based study of BRCA1 and BRCA2 mutations in 1035 unselected Finnish breast cancer patients. J Natl Cancer Inst 92, 1529-31 (2000).
41. Kilpivaara, O. et al. Correlation of CHEK2 protein expression and c.1100delC mutation status with tumor characteristics among unselected breast cancer patients. Int J Cancer 113, 575-80 (2005).
42. Eerola, H., Blomqvist, C., Pukkala, E., Pyrhonen, S. & Nevanlinna, H. Familial breast cancer in southern Finland: how prevalent are breast cancer families and can we trust the family history reported by patients? Eur J Cancer 36, 1143-8 (2000).
43. Vehmanen, P. et al. Low proportion of BRCA1 and BRCA2 mutations in Finnish breast cancer families: evidence for additional susceptibility genes. Hum Mol Genet 6, 2309-15 (1997).
44. Vahteristo, P. et al. A CHEK2 genetic variant contributing to a substantial fraction of familial breast cancer. Am J Hum Genet 71, 432-8 (2002).
45. Vahteristo, P., Eerola, H., Tamminen, A., Blomqvist, C. & Nevanlinna, H. A probability model for predicting BRCA1 and BRCA2 mutations in breast and breast-ovarian cancer families. Br J Cancer 84, 704-8 (2001).
46. Weinmann, A.S. & Famham, P.J. Identification of unknown target genes of human transcription factors using chromatin immunoprecipitation. Methods 26, 37-47 (2002).
47. Wells, J. & Farnham, P. J. Characterizing transcription factor binding sites using formaldehyde crosslinking and immunoprecipitation. Methods 26, 48-56 (2002).

1. An isolated nucleic acid molecule of less than 100, 50 or 30 nucleotides or base pairs comprising the sequence 5′ RRRCWWGYYYRRRCWWTYYY-3′_ (SEQ ID NO: 1) or its complement.

2. An isolated nucleic acid molecule of between 25, 50 or 100 and 300 nucleotides or base pairs comprising the sequence 5′ RRRCWWGYYYRRRCWWTYYY-3′_ (SEQ ID NO: 1) or 5′ RRRCWWGYYYRRRCWWGYYY-3′ (SEQ ID NO: 5) and capable of hybridising to or having at least 50, 60, 70, 80, 90 or 95% identity with, the region encompassing the p53 binding motif within the third intron of the PRKAG2 gene or its complement that can be amplified from human genomic DNA using the PCR primers 5′-TAGGAGACCTGGGGGACTTT-3′ (SEQ ID NO: 2) and 5′-CAGGCATCTCGAAGAGATCA-3′ (SEQ ID NO: 3).

3. The isolated nucleic acid molecule of claim 1 that is capable of hybridising to or having at least 50, 60, 70, 80, 90 or 95% identity with, the region encompassing the p53 binding motif within the third intron of the PRKAG2 gene or its complement that can be amplified from human genomic DNA using the PCR primers 5′-TAGGAGACCTGGGGGACTTT-3′ (SEQ ID NO: 2) and 5′-CAGGCATCTCGAAGAGATCA-3′ (SEQ ID NO: 3).

4. A vector comprising a nucleic acid according to claim 1.

5. An isolated nucleic acid having the sequence 5′-TAGGAGACCTGGGGGACTTT-3′ (SEQ ID NO: 7); 5′-CAGGCATCTCGAAGAGATCA-3′_ (SEQ ID NO: 8); 5′-CCATCCTGCCTGAGCATGTCTGAAC (SEQ ID NO: 9); or CCGGCTTTGCCAGACAATTGG (SEQ ID NO: 10).

6. The nucleic acid as defined in claim 5 for use in diagnosis.

7. A method for aiding assessment of a patient's risk of developing cancer, or likely severity or likelihood of progression of cancer, or aiding in selection of a cancer treatment regime for the patient, or aiding in assessment of a cancer treatment regime, the method comprising determining the patient's genotype for a p53 binding motif within the PRKAG2 gene.

8. The method of claim 7 wherein the p53 binding motif is within the third intron of the PRKAG2 gene.

9. The method of claim 7, wherein method comprises determining the presence or absence of a single nucleotide polymorphism relative to the wild-type p53 binding motif within the third intron of the PRKAG2 gene.

10. The method according to claim 9 wherein the wild-type p53 binding motif within the third intron of the PRKAG2 gene comprises the sequence 5′-RRRCWWGYYYRRRCWWGYYY-3′ (SEQ ID NO: 5) and can be amplified from human genomic DNA using the PCR primers 5′-TAGGAGACCTGGGGGACTTT-3′ (SEQ ID NO: 2) and 5′-CAGGCATCTCGAAGAGATCA-3′ (SEQ ID NO: 3).

11. The method according to claim 9, wherein the single nucleotide polymorphism is the substitution of the G residue marked with a * within the sequence 5′ RRRCWWGYYYRRRCWWG*YYY-3′ (SEQ ID NO: 11), for example by a T residue.

12. The method according to claim 9, wherein the single nucleotide polymorphism is at locus rs1860746 of the dbSNP public database.

13. The method according to ay one of claim 7 wherein sequencing, primer extension, allele-specific PCR or TaqMan assay is used in determining the patient's genotype for a p53 binding motif within the PRKAG2 gene.

14. A method for aiding assessment of a patient's risk of developing cancer, or likely severity or likelihood of progression of cancer, or aiding in selection of a cancer treatment regime for the patient, or aiding in assessment of a cancer treatment regime, the method comprising determining the patient's AMPK protein levels, phosphorylation levels, catalytic activity or mRNA levels.

15. The method according to claim 7 wherein the cancer is breast cancer or endometrial cancer.

16. A molecule comprising a nucleic acid molecule according to claim 1 and a detectable moiety.

17. The molecule of claim 16 wherein the detectable moiety is a fluorophore or a radioisotope.

18. A kit for aiding assessment of a patient's risk of developing cancer, or likely severity or likelihood of progression of cancer, or aiding in selection of a cancer treatment regime for the patient, or aiding in assessment of a cancer treatment regime, the kit comprising a nucleic acid molecule according to claim 1 and a package insert containing instructions using the kit.

19. Use of cells of a lymphoblastoid cell line for studying p53 signalling or in performing a screen for identifying modulators of p53 signalling.

20. A method for studying p53 signalling or for performing a screen for identifying modulators of p53 signalling comprising the step of assessing cells of a lymphoblastoid cell line.

21. A vector comprising a nucleic acid according to claim 2.

22. The method according to claim 14 wherein the cancer is breast cancer or endometrial cancer.

23. A molecule comprising a nucleic acid molecule according to claim 4 and a detectable moiety.

24. A kit for aiding assessment of a patient's risk of developing cancer, or likely severity or likelihood of progression of cancer, or aiding in selection of a cancer treatment regime for the patient, or aiding in assessment of a cancer treatment regime, the kit comprising a molecule according to claim 16 and a package insert containing instructions using the kit.


SEQUENCE LISTING (20 sequences)

SEQ ID NO: 1 - DNA, 20 bp, Homo sapiens
rrrcwwgyyyrrrcwwtyyy

SEQ ID NO: 2 - DNA, 20 bp, Artificial Sequence
  Description of Artificial Sequence Synthetic primer
taggagacctgggggacttt

SEQ ID NO: 3 - DNA, 20 bp, Artificial Sequence
  Description of Artificial Sequence Synthetic primer
caggcatctcgaagagatca

SEQ ID NO: 4 - DNA, 142 bp, Homo sapiens
ccatcctgcctgagcatgtctgaacatgttcttaggtcaggactagagttcgagatttcagaaatgtcattctaaccttgatctcttcgagatgcctgtttataacacagcatcgttcatgccaattgtctggcaaagccgg

SEQ ID NO: 5 - DNA, 20 bp, Homo sapiens
rrrcwwgyyyrrrcwwgyyy

SEQ ID NO: 6 - DNA, 226 bp, Homo sapiens
taggagacctgggggactttcatactctcagctgatgccagggtgcccagtgagcaggggaaaggcttcctggccctggcggcaggatggggccagaatattcctgggcaggagcccccccaggtggcccatcctgcctgagcatgtctgaacatgttcttaggtcaggactagagttcgagatttcagaaatgtcattctaaccttgatctcttcgagatgcctg

SEQ ID NO: 7 - DNA, 20 bp, Homo sapiens
taggagacctgggggacttt

SEQ ID NO: 8 - DNA, 20 bp, Homo sapiens
caggcatctcgaagagatca

SEQ ID NO: 9 - DNA, 25 bp, Homo sapiens
ccatcctgcctgagcatgtctgaac

SEQ ID NO: 10 - DNA, 21 bp, Homo sapiens
ccggctttgccagacaattgg

SEQ ID NO: 11 - DNA, 20 bp, Homo sapiens
rrrcwwgyyyrrrcwwkyyy

SEQ ID NO: 12 - DNA, 25 bp, Artificial Sequence
  Description of Artificial Sequence Synthetic primer
ccatcctgcctgagcatgtctgaac

SEQ ID NO: 13 - DNA, 21 bp, Artificial Sequence
  Description of Artificial Sequence Synthetic primer
ccggctttgccagacaattgg

SEQ ID NO: 14 - DNA, 25 bp, Artificial Sequence
  Description of Artificial Sequence Synthetic primer
caggctgtggctctgattggctttc

SEQ ID NO: 15 - DNA, 30 bp, Artificial Sequence
  Description of Artificial Sequence Synthetic primer
gctggcagatcacataccctgttcagagta

SEQ ID NO: 16 - DNA, 24 bp, Artificial Sequence
  Description of Artificial Sequence Synthetic primer
acccacactgtgcccatctacgag

SEQ ID NO: 17 - DNA, 26 bp, Artificial Sequence
  Description of Artificial Sequence Synthetic primer
tctccttaatgtcacgcacgatttcc

SEQ ID NO: 18 - DNA, 23 bp, Artificial Sequence
  Description of Artificial Sequence Synthetic primer
gtttcccctggaatcctataagc

SEQ ID NO: 19 - DNA, 21 bp, Artificial Sequence
  Description of Artificial Sequence Synthetic primer
cgaggcatagatgcgattctc

SEQ ID NO: 20 - DNA, 14 bp, Artificial Sequence
  Description of Artificial Sequence Synthetic probe
cgagcctgaacggt